(19) United States
(12) Patent Application Publication
(10) Pub. No.: US 2022/0108775 A1
(43) Pub. Date: Apr. 7, 2022

(54) TECHNIQUES FOR UPDATING A HEALTH-RELATED RECORD OF A USER OF AN INPUT/OUTPUT DEVICE

(71) Applicant: Intuition Robotics, Ltd, Ramat-Gan (IL)

(72) Inventors: Shay ZWEIG, Harel (IL); Chen SORIAS, Kibbutz Zikim (IL); Itai MENDELSOHN, Tel Aviv (IL); Roy AMIR, Mikhmoret (IL); Dor SKULER, Oranit (IL); Liran DORON, Kiryat Ono (IL)

(73) Assignee: Intuition Robotics, Ltd, Ramat-Gan (IL)

(21) Appl. No.: 17/495,191

(22) Filed: Oct. 6, 2021

Related U.S. Application Data
(60) Provisional application No. 63/088,082, filed on Oct. 6, 2020.

(51) Int. Cl. G16H 10/20; G16H 10/60; G06N 20/00

(52) U.S. Cl. CPC G16H 10/20; G06N 20/00; G16H 10/60

(57) ABSTRACT

A method for updating electronic records of a user including: creating a policy based on historic states of a user and feedback data captured by an input/output (I/O) device in response to projection of a plurality of health-related questions, wherein the policy includes a plurality of rules for determining customized questions for the user and a plurality of rules for determining plans for projecting customized questions to the user; determining at least one customized question for the user based on the policy and a health-related data of the user; determining a plan for projecting the at least one customized question to the user based on the policy and a current state of the user; projecting the at least one customized question via the I/O device; and updating an electronic record based on a response by the user to the at least one customized question captured via the I/O device.

### CROSS-REFERENCE TO RELATED APPLICATIONS

This application claims the benefit of U.S. Provisional Application No. 63/088,082, filed on Oct. 6, 2020, the contents of which are hereby incorporated by reference.

### TECHNICAL FIELD

The disclosure generally relates to digital assistants operated in an input/output (I/O) device, and more specifically to techniques for updating a health-related record of a user.

### BACKGROUND

As manufacturers improve the functionality of devices such as vehicles, computers, mobile phones, appliances, and the like, through the addition of digital features, manufacturers and end-users may desire enhanced device functionalities. The manufacturers, as well as the relevant end-users, may desire digital features which improve user experiences, interactions, and features which provide for greater connectivity. Certain manufacturers may include device-specific features, such as setup wizards and virtual assistants, to improve device utility and functionality. Further, certain software packages may be added to devices, either at the point of manufacture, or by a user after purchase, to improve device functionality. Such software packages may provide functionalities including, as examples, a computer system's voice control, facial recognition, biometric authentication, and the like.

While the features and functionalities described hereinabove provide for certain enhancements to a user's experience when interacting with a device, the same features and functionalities, as may be added to a device by a user or manufacturer, fail to include certain aspects which may allow for a further-enhanced user experience. First, certain currently-implemented digital assistants and other user experience features may fail to provide for adaptive adjustment of the operation of the assistant or feature. For example, a digital assistant configured to play music may be programmed to use a specific type of music streaming services, thereby limiting the user experience. In addition, certain currently-implemented digital assistants and other user experience features may fail to provide for adjustment of assistant or feature operation based on context or environmental data. As an example, a digital assistant may be configured to present reminders to take vitamins at a certain time. However, such reminder may be inappropriate and disturbing when the user is surrounded by guests or at an important meeting.

Such adaptive adjustment of operation of the assistant or feature may be particularly important and useful for assisting users in health-related operations that are closely related to the well-being of a user. As an example, when there are two users in the same room with different health conditions, a suggestion regarding one health condition must be direct to the correct user. Furthermore, an appropriate time and means of presenting such suggestions is important for such personal and private information. However, the currently-implemented digital assistants often lack such adaptiveness for each of the multiple users. As such, users may not benefit from policies or actions performed or suggested by the digital assistants, and eventually abandon the usage of such device.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for updating electronic records of a user via an input/output device. The method comprises: creating a policy based on historic states of a user and feedback data captured by an input/output (I/O) device in response to projection of a plurality of health-related questions, wherein the policy includes a plurality of rules for determining customized questions for the user and a plurality of rules for determining plans for projecting customized questions to the user; determining at least one customized question for the user based on the policy and a health-related data of the user; determining a plan for projecting the at least one customized question to the user based on the policy and a current state of the user; projecting the at least one customized question via the I/O device; and updating an electronic record based on a response by the user to the at least one customized question captured via the I/O device.

Certain embodiments disclosed herein also include a non-transitory computer readable medium having stored thereon causing a processing circuitry to execute a process, the process comprising: creating a policy based on historic states of a user and feedback data captured by an input/output (I/O) device in response to projection of a plurality of health-related questions, wherein the policy includes a plurality of rules for determining customized questions for the user and a plurality of rules for determining plans for projecting customized questions to the user; determining at least one customized question for the user based on the policy and a health-related data of the user; determining a plan for projecting the at least one customized question to the user based on the policy and a current state of the user; projecting the at least one customized question via the I/O device; and updating an electronic record based on a response by the user to the at least one customized question captured via the I/O device.

Certain embodiments disclosed herein also include a system for updating electronic records of a user via an input/output device. The system comprises: a processing circuitry; and a memory, the memory containing instructions that, when executed by the processing circuitry, configure the system to: create a policy based on historic states of a user and feedback data captured by an input/output (I/O) device in response to projection of a plurality of health-related questions, wherein the policy includes a plurality of rules for determining customized questions for the user and a plurality of rules for determining plans for projecting customized questions to the user; determine at least one customized question for the user based on the policy and a health-related data of the user; determine a plan for projecting the at least one customized question to the user based on the policy and a current state of the user; project the at least one customized question via the I/O device; and update an electronic record based on a response by the user to the at least one customized question captured via the I/O device.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram utilized to describe the various embodiments of the disclosure.

FIG. 2 is a block diagram of a controller, according to an embodiment.

FIG. 3 is a flowchart illustrating a method for customizing question policies and providing a customized health-related question to a user of a digital assistant according to an embodiment.

### DETAILED DESCRIPTION

The embodiments disclosed by the disclosure are only examples of the many possible advantageous uses and implementations of the innovative teachings presented herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality. In the drawings, like numerals refer to like parts through several views.

The embodiments disclosed herein provide techniques for efficiently and accurately updating the health-related record of a user of a digital assistant by incorporating one or more customized question policies. A customized question policy is created for each of the multiple users of the digital assistant based on their respective input data, and thereby providing a customized health-related question and a plan that is tailored to the specific user. Moreover, the customized question policies may be further modified to more accurately serve each user in different circumstances based on analyses of the sensor data, as may relate to a user, the conditions of the user's environment, and the like.

It should be appreciated that collecting accurate answers pertaining to health-related questions may be critical in monitoring and tracking a user's health condition and well-being. In order to receive meaningful answers (i.e., that are true reflections of their condition) from the user, presenting an applicable question using a desirable method and at the right time is highly desired. To this end, in an embodiment, user's current state is determined to generate, together with a customized policy, a plan for projecting a health-related question to the user. In an embodiment, the current state is generated based on real-time data of the user and the user's environment to define whether the user is, for example, awake, sleeping, angry, and the like, and the user's environment, for example, of being alone, with people, time, weather, and more. Here, the customized policy is dynamically modified by the digital assistant based on input data including, but not limited to, historic states and user's reaction as feedback data. In an embodiment, the customized policy is not preset for each user, but continuously modified to improve the accuracy and better reflect the needs and preferences of each user of the digital assistant. Such improvement in policy accuracy reduces false output (i.e., non-applicable question and/or at inappropriate times) from the digital assistant and instead increases frequency (or collection) of valuable responses from the users that precisely reflect their health conditions.

The embodiments disclosed herein provide advantageous objective rules-based analyses of the input data to increase consistency and accuracy of the policy for interacting with users of the digital assistant. While individuals (e.g., spouse, doctor, etc.) may present and update a health-related record, policy-related decisions such as, but not limited to, when and/or how to ask question, as wells as the analyses of sensor data can be highly subjective. In this approach, each individual that participates may subjectively generate a plan to project the question based on their “feeling” of the user's current state and response to previously presented questions, where the same user response may provide a different “feeling” for each individual to result inconsistent decision and output. However, a digital assistant adopting the customized question policy provides rules defined by, for example, weights, scores, ranking, and the like of certain parameters, to objectively analyze the user input data. In an embodiment, such objectively defined parameters are utilized to further modify and improve the customized question policy for the user. More specifically, the customized question policy may provide a more accurate and consistent presentation of the health-related question to each user; and in return the user's health-related records may be efficiently updated through accurate and continuous responses to the projected customized health-related questions.

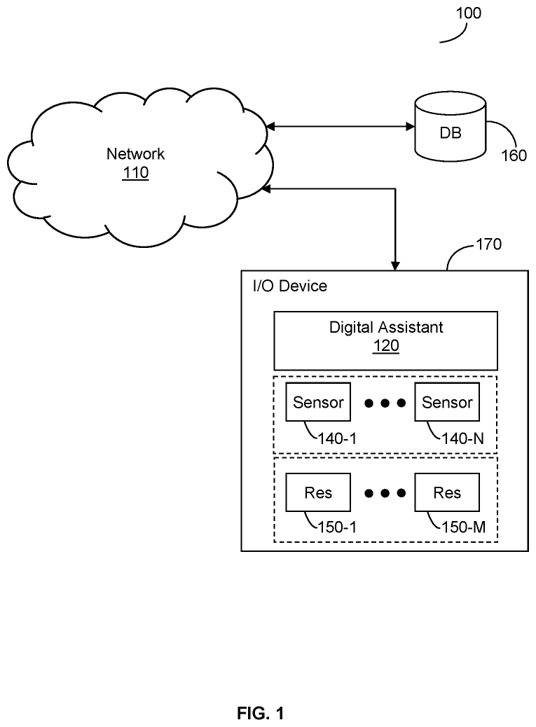

FIG. 1 is an example network diagram 100 utilized to describe the various disclosed embodiments. The network diagram 100 includes an input/output (I/O) device 170 operating a digital assistant 120. In some embodiments, the digital assistant 120 is further connected to a network 110 to allow some processing of a remote server (e.g., a cloud server). The network 110 may provide for communication between the elements shown in the network diagram 100. The network 110 may be, but is not limited to, a local area network (LAN), a wide area network (WAN), a metro area network (MAN), the Internet, a wireless, cellular, or wired network, and the like, and any combination thereof.

In an embodiment, the digital assistant 120 may be connected to, or implemented on, the I/O device 170. The I/O device 170 may be, for example and without limitation, a robot, a social robot, a service robot, a smart TV, a smartphone, a wearable device, a vehicle, a computer, a smart appliance, and the like.

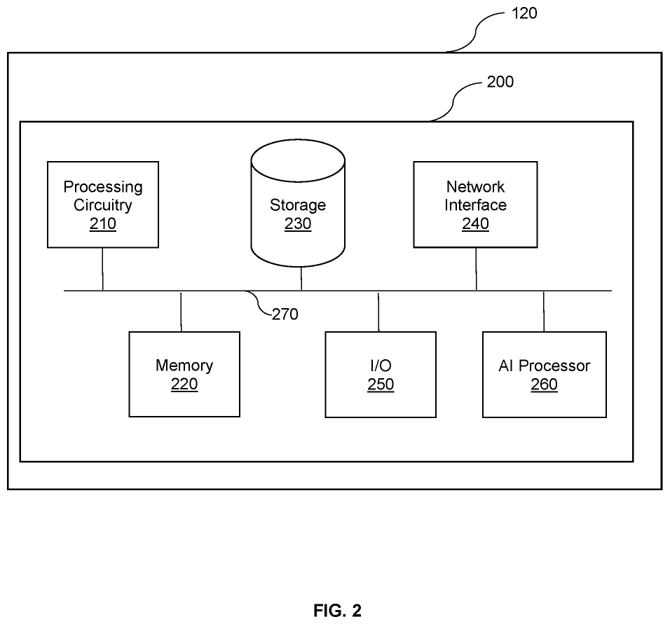

The digital assistant 120 may be realized in software, firmware, hardware, and any combination thereof. An example block diagram of a controller that may execute the processes of the digital assistant 120 is provided in FIG. 2. The digital assistant 120 is configured to process sensor data collected by one or more sensors, 140-1 to 140-N, where N is an integer equal to or greater than 1 (hereinafter referred to as “sensor” 140 or “sensors” 140 for simplicity) and one or more resources 150-1 to 150-M, where M is an integer equal to or greater than 1 (hereinafter referred to as “resource” 150 or “resources” 150 for simplicity). The resources 150 may include, for example, electro-mechanical elements, display units, speakers, and the like. In an embodiment, the resources 150 may include sensors 140 as well. The sensors 140 and the resources 150 are included in the I/O device 170.

The sensors 140 may include input devices, such as various sensors, detectors, microphones, touch sensors, movement detectors, cameras, and the like. Any of the sensors 140 may be, but are not necessarily, communicatively, or otherwise connected to the digital assistant 120 (such connection is not illustrated in FIG. 1 for the sake of simplicity and without limitation on the disclosed embodiments). The sensors 140 may be configured to sense signals received from a user interacting with the I/O device 170 or the digital assistant 120, signals received from the environment surrounding the user, and the like. In an embodiment, the sensors 140 may be implemented as virtual sensors that receive inputs from online services, for example, the weather forecast, a user's calendar, and the like.

In an embodiment, a database (DB) 160 may be utilized. The database 160 may be part of the I/O device 170 (e.g., within a storage device not shown), or may be separate from the I/O device 170 and connected thereto via the network 110. The database 160 may be utilized for storing, for example, historical data, health-related data about one or more users, related policies, diagnosis protocols, monitoring protocols, and the like, as well as any combination thereof.

FIG. 2 is an example block diagram of a controller 200 acting as a hardware layer of a digital assistant 120, according to an embodiment. The controller 200 includes a processing circuitry 210 that is configured to receive data, analyze data, generate outputs, and the like, as further described hereinbelow. The processing circuitry 210 may be realized as one or more hardware logic components and circuits. For example, and without limitation, illustrative types of hardware logic components that can be used include field programmable gate arrays (FPGAs), application-specific integrated circuits (ASICs), application-specific standard products (ASSPs), system-on-a-chip systems (SOCs), general-purpose microprocessors, microcontrollers, digital signal processors (DSPs), and the like, or any other hardware logic components that can perform calculations or other manipulations of information.

The controller 200 further includes a memory 220. The memory 220 may contain therein instructions that, when executed by the processing circuitry 210, can cause the controller 200 to execute actions as further described hereinbelow. The memory 220 may further store therein information, e.g., data associated with one or more users, historical data, and health-related data about one or more users, users' preferences and related policies, diagnosis protocols, monitoring protocols, and the like.

The storage 230 may be magnetic storage, optical storage, and the like, and may be realized, for example, as a flash memory or other memory technology, or any other medium which can be used to store the desired information.

In an embodiment, the controller 200 includes a network interface 240 that is configured to connect to a network, e.g., the network 110 of FIG. 1. The network interface 240 may include, but is not limited to, a wired interface (e.g., an Ethernet port) or a wireless port (e.g., an 802.11 compliant Wi-Fi card), configured to connect to a network (not shown).

The controller 200 further includes an input/output (I/O) interface 250 configured to control the resources (150, FIG. 1) which are connected to the digital assistant 120. In an embodiment, the I/O interface 250 is configured to receive one or more signals captured by the sensors (140, FIG. 1) of the digital assistant (120, FIG. 1) and to send such signals to the processing circuitry 210 for analysis. In an embodiment, the I/O interface 250 is configured to analyze the signals captured by the sensors 140, detectors, and the like. In a further embodiment, the I/O interface 250 is configured to send one or more commands to one or more of the resources 150 for executing one or more plans (e.g., actions) of the digital assistant 120, as further discussed herein below. A plan may include, for example, presenting a customized health-related question according to a user's policy. In further embodiment, the components of the controller 200 are connected via a bus 270.

In some configurations, the controller 200 may further include an artificial intelligence (AI) processor 260. The AI processor 260 may be realized as one or more hardware logic components and circuits, including graphics processing units (GPUs), tensor processing units (TPUs), neural processing units, vision processing units (VPU), reconfigurable field-programmable gate arrays (FPGA), and the like. The AI processor 260 is configured to perform, for example, machine learning based on sensory inputs received from the I/O interface 250, where the I/O interface 250 receives input data, such as sensory inputs, from the sensors 140.

In an embodiment, the controller 200 is configured to collect a set of health-related data about the user. It should be noted that the user may be a user of a digital assistant (e.g., the digital assistant 120). The digital assistant realized by the controller 200 may be associated with multiple users and collect health-related data for each of the multiple users. Health-related data may include current and historic information that is indicative to the user's health condition, chronic diseases, mental health issues, and so on.

In an example, the health-related data may indicate that the user suffers from diabetes, that the user should take certain prescription medications, and so on. In an embodiment, the health-related data may include information that is provided directly by the user when the user communicates with the digital assistant 120. In another embodiment, the health-related data may be collected by the digital assistant 120 when the user talks about her or his medical condition with a friend near the digital assistant 120. Thus, although the user was not actively asked to provide health-related data, the digital assistant 120 (i.e., the controller 200) collects the health-related data without bothering the user. Collecting the health-related data may be achieved using one or more sensors (e.g., the sensors 140). In another embodiment, health-related data of the user may be collected by the digital assistant 120 from one or more data sources, such as, websites, social media, user's calendar, online forums, and the like. The collected health-related data may be stored in a database (e.g., the database 160).

In further embodiment, the health-related data of the user may be collected by a medical care team (e.g., a doctor, a nurse, etc.) or any other authorized entity (e.g., a relative), and thereafter may be inputted and stored in a database (e.g., the database 160) for further usage. It should be noted that the health-related data may be inputted by, for example, the medical care team using one or more interfaces (such as the I/O interface 250 and the network interface 240) of the controller 200. As an example, when the user's doctor visits the user at the user's home, the I/O interface 250 can collect and input the health-related data of the user. As another example, when the user meets the doctor at the doctor's clinic, the doctor may input the health-related data of the user to the user's medical record which may be communicatively connected to the digital assistant 120 through the network interface 240.

In a further embodiment, the controller 200 is configured to collect a set of historical data about one or more users. It should be noted that the user may be a user of a digital assistant (e.g., the digital assistant 120). The digital assistant may be associate with multiple users and collect historical data for each of the multiple users. Historical data may refer to the user's preference, behavioral patterns, routines, and the like. As a non-limiting example, the historical data may indicate that the user takes a certain medication on a daily basis and sleeps at 11 pm.

In an embodiment, the controller 200, when executing the digital assistant 120, is configured to analyze the health-related data and historical data by applying at least one algorithm, such as a machine learning algorithm, to create a policy for the respective user from whom the data was collected. In an example embodiment, the machine learning may be a supervised machine learning trained over the health-related data and historical data related to different users. The generated policy may include a set of rules to be used by the I/O device 170 when interacting with a respective user. The set of rules may include specific guidelines indicating how to determine a customized health-related question and which plan is to be executed for projecting the customized health-related question to the user of the I/O device 170. It should be particularly noted that new policies can be created from a previously created policy based on a user response (e.g., feedback data) to the executed plan herein discussed further below.

In an embodiment, the controller 200, when executing the digital assistant 120, collects at least one user response from the user with respect to the at least one customized question as well as a user historic state at the time of response. The user response (e.g., feedback data) may include the user's reaction to the questions presented by the digital assistant 120. The user response may include a limited set of possible answers such as “yes”/“no” answers and a free-form answers. The user response (e.g., feedback data) may be stored for further usage in a database (e.g., the database 160). The user historic state may indicate the condition of the user and the condition of the environment near the user when the customized question is projected. The historic state may indicate whether, for example, the user was injured, not feeling good, crying, stressed, angry, sleeping, and so on. The historic state may further indicate the time, weather, number of people in the room, people identity, and so on. when the customized health-related question was presented. As noted above, in an embodiment, the collected feedback data and historic state is applied to the previously determined policy to create a new policy. That is, set of data including, but not limited to, health-related data, historical data, feedback data, and historic states may be applied to at least one algorithm, such as machine learning algorithm, to generate a policy for each of the users of the I/O device 170. It should be noted that the new policy may include a new set of rules to execute a plan for projecting a customized health-related question to the respective user of the I/O device 170 (i.e., digital assistant 12). That is, the policy created based on user responses and historic states may become a closer reflection of each user's preference and needs pertaining to their health.

In an embodiment, the controller 200, when executing the digital assistant 120, is configured to determine a customized health-related question for the user of the digital assistant 120 based on rules of the policy created for the respective user. In an embodiment, the customized health-related question may be extracted from a predetermined protocol (such as a diagnosis protocol or a monitoring protocol) of a plurality of predetermined protocols. A diagnosis protocol or a monitoring protocol may include a set of questions designed to enable a better understanding of the user's (or patient's) health condition. There may be multiple diagnosis protocols or monitoring protocols where each of the protocols is specifically utilized for diagnosing and/or monitoring the development of a particular medical condition, such as, overweight, diabetes, hypertension, schizophrenia, backache, and the like.

It should be noted that the customized health-related question is selected based on the information (e.g., health-related data) that is actively or passively collected by the digital assistant 120. As an example, when the health-related dataset indicates that the user suffers from a broken leg the controller 200 may be configured to select questions from a monitoring protocol that is relevant to monitoring the healing process of the user's broken leg.

In an embodiment, the controller 200, when executing the digital assistant 120, is configured to present the determined customized health-related question to the user. Presenting the customized health-related question may be performed using one or more resources (e.g., the resources 150) that are communicatively connected to and controlled by the digital assistant 120. Such resources may be for example, a speaker, a display unit, a smartphone that is communicatively connected to the digital assistant 120, and so on. In an embodiment, the plan for presenting (or projecting) the customized health-related question may be determined by rules of the user policy.

In an embodiment, prior to presenting the customized health-related question to the user, the controller 200 is configured to collect a set of real-time data that is associated with a specific user and an environment in a predetermined proximity to the user. The real-time data may be sensed or otherwise collected using one or more sensors (e.g., the sensors 140, FIG. 1), and may indicate, for example, the user's mood, the specific location of the user, whether the user is awake or asleep, and more. Furthermore, the controller 200 may be configured to collect real-time data about the user's environment, such as the number of people near the user, the time, the weather, etc. The real-time data is collected and analyzed in order to determine a current state of the user and the environment in a predetermined proximity to the user. The current state may reflect the condition of the user and the condition of the environment near the user in real-time, or near real-time. The current state may indicate whether, for example, the user is injured, not feeling good, crying, stressed, angry, sleeping, and so on. The current state may further indicate the current time, weather, number of people in the room, people identity, and so on.

In an embodiment, the controller 200, when executing the digital assistant 120, is configured to determine and execute a plan for projecting the customized health-related question to the user. The plan may be generated based on the determined current state and the policy of the respective user. The plan may be personalized for the user at the current state based on a plurality of rules of the respective policy. That is, plan based on the user specific policy may enable the digital assistant 120 to take different communication approaches with different user. For example, a first user may prefer a more sensitive approach compared to a second user that may prefer a more cynical approach. Moreover, even for the same user, different plans can be executed depending on the current state of the user. In an example embodiment, a health-related question may or may not presented, and if projected, in different approach such as, but not limited to, text, audio, video, tone of audio, sentence structure, details in question, and more. As an example, when the current state indicates that the user is asleep and the time is 3 am, it may not be desirable to present a question to the user however, if the user is in the kitchen, the time is 10 am and the user is alone it may be desirable to present a question to the user. According to the same example, in case the current state indicates that the user has company (so the user is not alone at home), it may not be desirable to present a personal question to the public. In one embodiment, the controller 200, when executing the digital assistant 120, may be configured to adjust or change the determined customized health-related question based on the determined current state. That is, the determined current state may affect the election and/or modification of the at least one health-related question. For example, in case the selected first question is: “have you checked your blood pressure this morning as you usually do?” however, the current state indicates that an unfamiliar person is located near the user, the controller 200 may adjust the question to be: “have you performed your morning ritual?”. According to the same example, if the other person with the user is identified as the user's child, the controller 200 may not change or adjust the original question.

In an embodiment, the controller 200, when executing the digital assistant 120, is configured to update a health-related record that is associated with the user. The health-related record may be an electronic file utilized for storing user's health-related data, monitoring, and tracking the user's medical condition, detect patterns of health issues, and more. In an embodiment, the health-related electronic record is updated periodically. As an example, a user that suffers from hypertension may be frequently asked by the digital assistant 120 about the following possible symptoms: vision problems, severe headaches, difficulty breathing, Irregular heartbeat, blood in the urine, and so on. Based on the user response, the health-related record is updated. The periodically updated health-related electronic record enables tracking and monitoring of the user's health condition across time. In an embodiment, the controller 200, when executing the digital assistant 120, is configured to generate a notification or an alert indicating that the health-related electronic record of the user has been updated. In addition, the notification or alert may include the new and updated health-related dataset. In a further embodiment, the controller 200 may be configured to send the new and updated data to, for example, a predefined server, a predefined end-point device, and the like, that is associated with, for example, a third party. For that matter, the controller 200 may be configured to send the new health-related dataset using an application programming interface (API) of a third party.

In an embodiment, the controller 200, when executing the digital assistant 120, may be configured to generate, based on the updated health-related record, at least one incentive for the user to keep providing feedback to at least a second question. An incentive may include an electronic report that includes analytics showing how the user's answers (feedback data collected from the user) contributed to the process of monitoring and tracking the user's health condition. The incentive may include for example, a visual representation of at least a portion of the health-related record, a sentence executed by the digital assistant 120 (i.e., by the controller 200), a combination thereof, and the like.

In a further embodiment, the user response (e.g., feedback data) that may be stored in a database (e.g., the database 160), may be analyzed and monitored by the controller 200. By analyzing and monitoring the user responses through time, health-related patterns and abnormal health-related behaviors may be detected. Detecting health-related information such as, but not limited it, health-related patterns, abnormal health-related behavior, and the like, may be achieved using, for example, one or more designated algorithms, such as machine learning algorithms, that are adapted to detect health-related patterns, abnormal health-related behavior, and so on. As a non-limiting example, a certain user always provides a similar answer to the question: “how did you sleep?”, however at some point the answer changes dramatically. According to the same example, due to the frequently asked question the controller 200 may be configured to detect that something is wrong, i.e., that an anomaly has been detected with respect to the user's sleeping habits.

In an embodiment, the controller 200, when executing the digital assistant 120, may be configured to generate a dashboard that includes at least the updated health-related electronic record of the user. The dashboard may be used for presenting a medical condition progress, monitoring unusual health-related parameters, and more. A health-related parameter may be, for example and without limitation, the user's blood pressure, sleep quality, and the like. In another embodiment, the controller 200 may be configured to generate at least one notification upon determination that at least one health-related parameter (e.g., blood pressure, sleep quality, etc.) crosses a predetermined threshold value. In a further embodiment, the controller 200, when executing the digital assistant 120, may be configured to send the generated dashboard including the most updated health-related record of the user and/or the at least one notification to a predefined electronic source. The predefined electronic source may be for example, a server, a personal computer, and the like. that is associated with, for example, a medical care team, a relative, and the like.

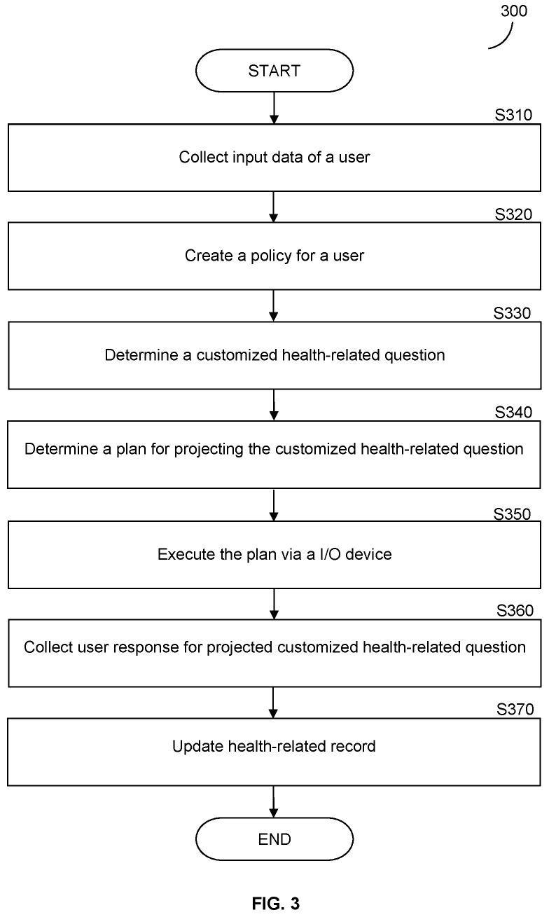

FIG. 3 shows an example flowchart 300 of a method for customizing question policies and providing a customized question to a user of a digital assistant according to an embodiment. The method described herein may be executed by the controller 200 that is further described herein above with respect to FIG. 2. As noted above, the controller 200 is a hardware layer running the digital assistant 120.

At S310, input data about a user is collected. The input data may include at least a health-related data and a historical data with respect to each of the users of the digital assistant 120. As described hereinabove, health-related data may indicate the user's health condition, chronic diseases, mental health issues, and the like. Historical data may include general historical information of the user, the user's habits, hobbies, preferences, patterns, and more. In further embodiment, a real-time data about the user and the environment in a predetermined proximity to the user is collected at the digital assistant 120 (or controller 200). The real-time data may be sensed or otherwise collected using one or more sensors (e.g., the sensors 140 shown in FIG. 1), and may indicate, for example, the user's mood, the specific location of the user, whether the user is awake or asleep, etc. In a further embodiment, the controller 200 may be configured to collect real-time data about the user's environment, such as the number of people near the user, the time, the weather, and more. Moreover, a feedback data is captured at the I/O device including the digital assistant 120. The feedback data includes user's response and state when a selected question is presented to the user.

At S320, a policy is created for the user. At least one algorithm, such as a machine learning algorithm, is applied to the input data and adapted to determine a policy for presenting a question to a user of the digital assistant 120. In an embodiment, the policy includes multiple rules for determining a customized health-related question and multiple rules for determining a plan for presenting the customized health-related question. In an embodiment, a brand-new policy is created for each of the users and stored for subsequent use. In an embodiment, the policy of the user may be adapted and personalized further by applying at least the feedback data of the user's response and historic state for the projected question. That is, a new policy may be created from the user's old policy. In this scenario, the policy for presenting a health-related question may become more tailored for the user's needs, preference, behavioral patterns, and the like to enable frequent monitoring of the user's medical condition. It should be noted that a policy may be created separately for each user of the digital assistant based on their respective input data collected.

At S330, a customized health-related question is determined based on the created policy and the health-related data of the user. The customized health-related question may be extracted from a predetermined protocol of a plurality of predetermined protocols (e.g., a diagnosis protocol, a monitoring protocol, etc.). In another embodiment, other input data, such as real-time data, historical data, and feedback data, may applied to determine a customized health-related question.

At S340, a plan for projecting the customized health-related question is determined based on the created policy and a current state of the user. In an embodiment, the current state may be generated using at least one algorithm, such as machine learning algorithm, that may be stored in a memory (e.g., memory 220, FIG. 2). The algorithm may facilitate determination of at least a current state of the user based on at least a portion of the input data. In a further embodiment, the algorithm may facilitate determination of a current state of the environment near the user (e.g., in a predetermined proximity to the user) based on at least a portion of the input of data. It should be noted that the collected set of data may be fed into the abovementioned algorithm, therefore allowing the algorithm to determine the current state. According to further embodiment, the collected set of data may be analyzed using, for example and without limitations, one or more computer vision techniques, audio signal processing techniques, machine learning techniques, and the like.

According to the embodiment, the policy adjusts the plans that are executed by the digital assistant 120 in order to tailor for the current state of the user. In an example embodiment, an executed plan of the policy may result in the digital assistant 120 staying silent (i.e., not asking the question) based on the determined current state of the user. In another example embodiment, a health-related question may be customized for the user using a soft tone when the time is 11 pm. It should be noted that the policy may include multiple rules related to the user's input data where certain parameters (or characteristics) may have more weight, priority over others to allow ranking, and thus objectively determining the plan.

At S350, the plan is executed to project the customized health-related question via an I/O device 170 to the user. In an embodiment, the customized health-related question may be presented through at least one of the resources (e.g., resource 150, FIG. 1). As noted above, personalizing the presentation of the customized health-related question may result in the digital agent communicating differently with different users.

At S360, the user response (e.g., feedback data) is collected from the user with respect to the presented health-related question. The user response includes the user's reaction to the questions presented via a I/O device 170. In an example embodiment, the feedback data may include, but not limited, silence, “yes” or “no” answer, free form answer, sound, movement, and more. In an embodiment, the feedback data is captured at the I/O deice 170 using at least one or more sensors (140, FIG. 1) and include, without limitation, images, video, audio signals, and the like.

At S370, the health-related record associated with the user is updated based on the user response to the customized health-related question. The health-related record may be an electronic file utilized for storing user's health related data, monitoring, and tracking the user's medical condition, detect patterns of health issues, and so on. In an example embodiment, the updated health-related records of the user may be sent to a predefined electronic source (e.g., a medical care team) such as a server, a personal computer, and the like. In another example embodiment, a dashboard may be generated and updated with any new development in the user's health condition. The dashboard and any update related thereto may be sent to the predetermined electronic source. For example, the dashboard may be presented on the I/O device 170 or on a remote terminal of the doctor. In an embodiment, S370 may further include generating and sending a notification indicating a change, above a predefined threshold value, in one or more health-related parameters to a predefined electronic source, as further discussed herein above with respect to FIG. 2.

The various disclosed embodiments are implemented as hardware, firmware, software, or any combination thereof. Moreover, the software is preferably implemented as an application program tangibly embodied on a program storage unit or computer readable medium. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such computer or processor is explicitly shown. In addition, various other peripheral units may be connected to the computer platform such as an additional data storage unit and a printing unit.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for updating electronic records of a user via an input/output device, comprising:
creating a policy based on historic states of a user and feedback data captured by an input/output (I/O) device in response to projection of a plurality of health-related questions, wherein the policy includes a plurality of rules for determining customized questions for the user and a plurality of rules for determining plans for projecting customized questions to the user;
determining at least one customized question for the user based on the policy and a health-related data of the user;
determining a plan for projecting the at least one customized question to the user based on the policy and a current state of the user;
projecting the at least one customized question via the I/O device; and
updating an electronic record based on a response by the user to the at least one customized question captured via the I/O device.

2. The method of claim 1, wherein the customized question is a customized health-related question, and wherein the electronic record is a health-related record.

3. The method of claim 1, further comprising:
applying a machine learning model trained to determine the current state based on a real-time data of the user and a real-time data of an environment in a predetermined proximity to the user in real time.

4. The method of claim 3, wherein the real-time data of the user and a real-time data of an environment is captured by at least one sensor of the I/O device.

5. The method of claim 2, further comprising:
storing the response by the user to the at least one customized health-related question as a second feedback data and the current state of the user as a second historic states in a memory; and
generating a second policy based on the stored second feedback data and second historic states of the user.

6. The method of claim 2, wherein the at least one customized health-related question is projected by at least one resource of the I/O device.

7. The method of claim 1, further comprising:
generating based on the updated electronic record at least one incentive for the user to keep providing feedback to at least a second question.

8. The method of claim 2, further comprising:
generating a dashboard that includes at least the updated health-related record to the user.

9. The method of claim 8, wherein the dashboard is presented on at least one of:
the I/O device and a remote terminal.

10. A non-transitory computer readable medium having stored thereon instructions for causing a processing circuitry to execute a process, the processing comprising:
creating a policy based on historic states of a user and feedback data captured by an input/output (I/O) device in response to projection of a plurality of health-related questions, wherein the policy includes a plurality of rules for determining customized questions for the user and a plurality of rules for determining plans for projecting customized questions to the user;
determining at least one customized question for the user based on the policy and a health-related data of the user;
determining a plan for projecting the at least one customized question to the user based on the policy and a current state of the user;
projecting the at least one customized question via the I/O device; and
updating an electronic record based on a response by the user to the at least one customized question captured via the I/O device.

11. A system for updating electronic records of a user via an input/output device, comprising
a processing circuitry; and
a memory, the memory containing instructions that, when executed by the processing circuitry, configure the system to:
create a policy based on historic states of a user and feedback data captured by an input/output (I/O) device in response to projection of a plurality of health-related questions, wherein the policy includes a plurality of rules for determining customized questions for the user and a plurality of rules for determining plans for projecting customized questions to the user;
determine at least one customized question for the user based on the policy and a health-related data of the user;
determine a plan for projecting the at least one customized question to the user based on the policy and a current state of the user;
project the at least one customized question via the I/O device; and
update an electronic record based on a response by the user to the at least one customized question captured via the I/O device.

12. The system of claim 11, wherein the customized question is a customized health-related question, and wherein the electronic record is a health-related record.

13. The system of claim 11, wherein the system is further configured to:
apply a machine learning model trained to determine the current state based on a real-time data of the user and a real-time data of an environment in a predetermined proximity to the user in real time.

14. The system of claim 13, wherein the real-time data of the user and a real-time data of an environment is captured by at least one sensor of the I/O device.

15. The system of claim 12, wherein the system is further configured to:
store the response by the user to the at least one customized health-related question as a second feedback data and the current state of the user as a second historic states in a memory; and
generate a second policy based on the stored second feedback data and second historic states of the user.

16. The system of claim 12, wherein the at least one customized health-related question is projected by at least one resource of the I/O device.

17. The system of claim 11, wherein the system is further configured to:
generate based on the updated electronic record at least one incentive for the user to keep providing feedback to at least a second question.

18. The system of claim 12, wherein the system is further configured to:
generate a dashboard that includes at least the updated health-related record to the user.

19. The system of claim 18, wherein the dashboard is presented on at least one of: the I/O device and a remote terminal.
